(19) United States
(12) Patent Application Publication
(10) Pub. No.: US 2010/0215765 A1
(43) Pub. Date: Aug. 26, 2010

(54) Antimicrobial Composition and a Method of Controlling Contamination and Infection Using Said Composition

(76) Inventors: Alyson Bexfield, Newcastle upon Tyne (GB); Alison Elizabeth Bond, Swansea (GB); Edward Dudley, Swansea (GB); Russell Peter Newton, Killay Swansea (GB); Yamni Nigam, Swansea (GB); Norman Arthur Ratcliffe, Swansea (GB)

Correspondence Address: WADDEY & PATTERSON, P.C., 1600 DIVISION STREET, SUITE 500, NASHVILLE, TN, 37203, US

(21) Appl. No.: 12/523,662

(22) PCT Filed: Jan. 17, 2008
(86) PCT No.: PCT/GB2008/000157

§ 371 Date: Apr. 21, 2010

(30) Foreign Application Priority Data
Jan. 18, 2007 (GB) 0700946.7

(51) Int. Cl. A61K 35/64; A61P 17/02; A61P 29/00; C07G 11/00

(52) U.S. Cl. 424/538; 568/300

(57) ABSTRACT

An antimicrobial composition having the empirical formula of C₁₀H₁₆N₆O₉.

The present invention relates to an antimicrobial composition and to methods of controlling contamination and infection using said composition.

Antimicrobial compositions are well known and can be used to treat a range of organisms including bacteria, fungi, yeast and protozoa. Antimicrobials can be obtained from a range of sources for example from plants, from extractions isolated from microbial cultures or from secretions from invertebrates such as insects. It is known that an extract from the larvae of the green-bottle fly (Lucilia sericata) has antimicrobial properties.

Also, many different substances have been proposed to be used in the healing of wounds. These substances include collagenase, streptodornase and streptokinase which are obtained from bacterial sources. Other substances include Bromelain, obtained from pineapples, Krill enzymes which are obtained from crustaceae and plasmin and trypsin, which are typically obtained from mammalian sources such as cattle.

A problem with many antibiotics or healing agents is that they are often only partially effective. In particular, in the case of antibiotics, as a result of widespread and indiscriminate use, microbes have become resistant to the microbial effects of such antibiotic compounds. Particular antibiotic resistant “superbugs” include methicillin-resistant Staphylococcus aureus (MRSA) and vancomycin-resistant Enterococcus (VRE). In the USA, vancomycin-resistant strains of Staphylococcus aureus have also been found, which illustrates that bacteria are becoming resistant to a greater range of antibiotics. If a person is infected with such resistant organisms healing can be very prolonged and costly. Also, as the bacteria have built up a resistance to most of the commonly used antibiotics then the infection may never be fully controlled, which may result in a patient having an amputation of the infected area. The cost of these more intense treatments increases the financial burden on health care and, in particular, State health care provision. It is estimated that the cost of treating wound infections to the UK National Health Service is at least £1 billion annually and the emergence of antibiotic resistant microorganisms contributes to these costs.

It is known that the treatment of wounds with larvae from green-bottle flies can provide an effective and simple way of cleansing infected and necrotic wounds. The application of sterile larvae from Lucilia sericata to an infected wound results in the removal of necrotic and sloughy tissue, disinfection and enhancement of the healing process. The process of applying sterile fly larvae to wounds is generally known as maggot therapy.

Although it is recognised that there is a degree of antimicrobial action involved in maggot therapy, there has been much dispute as to how this occurs. It is thought that the process may be by a mechanical action for example mechanical irrigation of the wound by the physical digestion of tissue by the larvae, together with ingestion of bacteria by the larvae. Alternative theories are that excretions/secretions (ES) produced by the larvae may contain factors that may disinfect the wound.

A problem with maggot therapy is that the application of live maggots to the body is not acceptable to some patients and clinicians. They have a fear that the maggots may burrow under the skin and some imagine that they can feel the maggots moving in situ or are afraid that they could escape from a wound dressing. Although none of these events actually occur, the perception of them occurring is a real deterrent to some people accepting maggot therapy.

Investigations have been made to establish how maggot therapy leads to antimicrobial activity but although some studies recognise that the extracorporeal excretions/secretions of maggots may contain an antibiotic, there is no indication of what this is or whether it could be isolated from the exudates from the maggots.

In WO 03/07654, which covers work done by The University of Nottingham in the UK, it has been shown that the secretions/excretions of Lucilia sericata have N-acyl homoserine lactone degrading activity, which may be due to a serine proteinase or glycosidase. However, the compositions discussed are made up of the complete excretion/secretion from the maggots, there is no purified component that has been isolated, which can be used as an antimicrobial composition in its own right. Further, WO 03/07654 states that excretions/secretions produced from L. sericata grown under sterile conditions have no antibiotic effect on the growth of either Gram+ve or Gram−ve bacteria (S. aureus and Ps. aeruginosa respectively). The excretions/secretions discussed are used with a conventional antibiotic such as tetracyline because the combined effect of the larval secretions and the antibiotic are greater than when they are used separately.

In DE 10149153 A1, DE 10138303 A1, DE 19901134 A1 it is described that enzymes or enzymatic extracts from whole body homogenates of the larvae or pupae of Sarcophaga or Lucilia can be applied in the treatment of acute or chronic wounds. However, for the compositions discussed there is no specific purified component that has been isolated, which can be purified and used as an antimicrobial in general. Again, having a complete exudate from maggots may be unacceptable to medical personnel and patients alike.

The present seeks to overcome the problems of the prior art by providing a defined antimicrobial composition having a powerful antimicrobial activity, in particular in relation to antibiotic resistant microbes.

It is to be understood that the term antimicrobial refers to compounds and combinations of compounds that have bacteriostatic as well as bacteriocidal properties and which could be administered to a human or an animal to inhibit microbial infection. Although the antimicrobial is particularly applicable to wounds infected with bacteria, it may be used to treat other infections, for example fungal infections or infections where there is a mixture of bacteria and other organisms. Also, the antimicrobial may be administered topically or systemically for the treatment or prevention of infection. The antimicrobial may also be used in other applications such as, but not limited to, the prevention of food spoilage, as a disinfectant, an antiseptic, surface cleaner or preservative.

In short, the current invention is concerned with a particular defined compound that has been isolated from maggots which has the unexpected effect of being an antimicrobial that can be used in a wide range of applications. Examples of these applications, which is not to be considered to be a limited list include use as an antimicrobial in the treatment of wounds, which can be either topically or intravenously administered or to a general antimicrobial that can prevent contamination in a wide range of situations.

According to a first aspect of the present invention there is provided an antimicrobial composition having the empirical formula C₁₀H₁₆N₆O₉. This composition has been named by the inventors as Seraticin™.

It is preferred that the antimicrobial composition is an isolate from excretions/secretions of fly larvae although it is envisaged that it may be isolated from other sources such as vertebrates or invertebrates.

Preferably, the antimicrobial composition is obtained from fly larvae of the species Lucilia sericata.

The invention also relates to a pharmaceutical composition, which comprises combining the fly larval composition of the invention into a suitable dosage form with a pharmaceutically suitable and physiologically tolerated carrier and, where appropriate, other suitable active substances, additives or excipients.

The invention also relates to the use of the compositions for producing pharmaceuticals for the therapy of superficial or deep chronic and acute wounds of any etiology.

The pharmaceuticals of the invention are usually applied topically but they may be applied intracorporally, such as intravenously, or by implants in the body or by a transdermal application or even by way of oral application.

Suitable pharmaceutical compositions for topical use on the skin are preferably in the form of a solution, suspension, dusting powder, liposomal formulations, gel, lotion, paste, spray or aerosol. Carriers which can also be used are polyethylene glycols, alcohols and combinations of two or more of these substances. The list can by no means be regarded as restrictive. The fly larval microbial extracts of the invention are present in a concentration of from 0.1% by weight to 100% by weight of the composition, for example 50-90%, or 40-80%, 30-70% or from 1.0% by weight to 60% by weight, depending on the extraction conditions.

Transdermal administration is also possible. Suitable pharmaceutical compositions for transdermal uses may be in the form of individual plasters which are suitable for long-term close contact with the patient's epidermis. Plasters of this type suitably contain the microbial composition of the invention in an optionally buffered aqueous solution, dissolved and/or dispersed in an adhesive or dispersed in a polymer. A suitable active substance concentration is from about 0.01% by weight to 75% by weight, preferably from 1% by weight to 70% by weight.

The antimicrobial of the invention can also be applied to the wound through wound coverings made of gauze, of alginates, of hydrocolloid materials, foams and silicone coverings, which have been coated, impregnated or treated with the composition and are therefore able to deliver the composition into or onto the wound or wound surface.

The composition of the invention may also be delivered as an oral pharmaceutical such as a tablet.

Suitable solid pharmaceutical forms are, for example, granules, powders, solutions, suspensions, emulsions or drops, and the products with protracted release of active substance, in the production of which conventional excipients or carriers are used. Excipients which are frequently used and which may be mentioned are magnesium carbonate, titanium dioxide, lactose, mannitol and other sugars, talc, milk protein, gelatine, starch, cellulose and derivatives thereof, polyethylene glycol and solvents such as, for example, sterile water and monohydric or polyhydric alcohols such as glycerol.

According to a further aspect, the invention relates to a pharmaceutical composition arranged to be administered intravenously.

The carrier composition may be any pharmaceutically acceptable carrier such as a solvent for antibiotics, chelating agents and pH buffering agents that will allow a therapeutic composition to be administered directly to the skin, wound or mucosa or systemically by, for example but not limited to, oral or intravenous administration. The carrier may also allow the use of the antimicrobial for other purposes, for example but not limited to, antiseptics, disinfectants and preservatives. Examples of carriers that can be used, which is not a limiting list, include water, saline, physiological saline, ointments, creams, oil in water emulsions, gels, solvents or combinations of solvents.

The treatment preparation also includes a pH buffering agent. The term pH buffering agent includes, for example, pharmaceutically acceptable organic or inorganic compounds or combinations of compounds that will maintain the pH of the treatment preparation within desired pH limits.

It is envisaged that the treatment preparation further includes a surfactant. Known surfactants, such as detergents, or anionic or ionic surfactant or a mixture thereof may be used. Surfactants are included to alter the surface tension of aqueous solutions containing the antimicrobial composition and therefore provide a treatment preparation that can be easily taken into the body.

It is envisaged that the carrier may include one or more of a pH buffering agent, a tissue growth promoting agent, an anti-inflammatory or a pain killer and a surfactant or any such agents which allow administration via, for example, oral, parenteral or intravenous methods.

The treatment preparation may also include a wound healing promoter such as Vitamin E which can assist in the skin around a wound healing by promoting tissue growth.

In addition the treatment preparation may include, such as, one or more anti-inflammatory agents for example a corticosteroid or agents that can reduce pain, such as morphine based substances. These inclusions are particularly useful if the patient is feeling uncomfortable because the wound damage is deep seated.

In addition to treatment of the body, the antimicrobial composition may be incorporated in a formulation to treat food to reduce the risk of microbial contamination.

Further the antimicrobial may be incorporated in a disinfectant to be used as a general surface cleaner, for example, hard surfaces in bathrooms, kitchens and on floors.

The antimicrobial composition may also be used as a general antiseptic, which can be used in a range of environments, not only in the house, but in hospitals, workplaces, in areas where other sterile environments are required.

The antimicrobial may be incorporated in for example antiseptic wipes or in misters or it can be used as a liquid or gel in the form of a surface cleaner.

Further, the composition claimed when used as a medicament may be provided in the form of a salt, an ester or pro drug derivative.

According to a further aspect there is provided a method of treating a wound infection, said method comprising contacting a wound with a therapeutic amount of an antimicrobial composition of the empirical formula C₁₀H₁₆N₆O₉. Furthermore, infection may be treated by, but not limited to, oral, intravenous, or parenteral administration of the antimicrobial composition.

Preferably, the antimicrobial composition is an isolate from the secretions/excretions of fly larvae.

It is envisaged that preferably the species of fly larvae is Lucilia sericata.

Preferably, the antimicrobial composition is included in a pharmaceutically acceptable carrier.

It is envisaged that the carrier may include one or more of a pH buffering agent, a tissue growth promoting agent and a surfactant.

Treatment is preferably oral, or failing that, parenteral.

According to yet further aspect of the invention, there is provided a wound dressing including an antimicrobial composition having the empirical formula of C₁₀H₁₆N₆O₉.

Preferably the wound dressing includes a treatment preparation including an antimicrobial composition of the empirical formula C₁₀H₁₆N₆O₉ and a carrier composition for the antimicrobial composition.

It is envisaged that the carrier may include one or more of a pH buffering agent, a tissue growth promoting agent, an anti-inflammatory or a pain killer and a surfactant.

Although separate aspects or embodiments of the invention have been described, the invention is intended to also cover combinations of those aspects or embodiments.

The following examples are provided to illustrate preferred embodiments and uses of the present invention but should not be construed to limit the scope of the invention.

### EXAMPLE 1

### Methods and Materials

### Fly Larvae

Sterile third instar larvae of Lucilia sericata were supplied by the Zoobiotic Limited, Bridgend, UK.

### Microorganisms

A range of Gram-positive and Gram-negative bacteria and fungi, including but not limited to Methicillin-resistant Staphylococcus aureus (MRSA) Staphylococcus Sp., Bacillus sp., Escherichia coli, Pseudomonas sp., Proteus sp., Enterococcus sp., Serratia sp., Candida sp. and Enterobacter sp were purchased from the National Collections of Industrial and Marine Bacteria (NCIMB) Aberdeen, UK or provided by the Pathology Laboratory of the Princess of Wales Hospital, Bridgend, UK.

### Collection of Larval Excretions/Secretions

Native excretions/secretions (nES) were collected by incubating third-instar larvae in a small quantity (200 μl g⁻¹ of larvae) of sterile Milli-Q ultra pure water (Millipore UK Ltd, Hertfordshire, UK) for 1 h at 30° C. in darkness. The sterile liquid was siphoned off from the containers and centrifuged at 10,000×g for 5 minutes to remove particulate material, after which the supernatant was retained at −20 to −80° C. for testing. Although third-instar larvae were used, it is possible to use first and second instar-larvae but third instar-larvae yielded increased antimicrobial activity and sample volume.

### Purification of the Supernatant

A pooled volume of nES was fractionated in an Amicon stirred ultrafiltration cell (Millipore UK Ltd) that was pressurised with N₂ gas. The Amicon filters generated fractions from the excretions/secretions of different molecular weights—(Bexfield, A., et al., Microbes and Infection 6, 1297-1304, -2004).

### Separation of Fractions

Fractions of maggot excretion/secretion filtrate having molecular weights of <500 Da were concentrated 10 times using an Eppendorf Speedvac vacuum centrifuge at 25-35° C. prior to any HPLC separation.

Initially, 100 μl of the concentrated <500 Da filtrate was injected onto a C30 HPLC column and guard column from YMC Europe GmbH (Germany) using a Vision Workstation (Applied Biosciences, U.K).

The mobile phase consisted of an aqueous mobile phase A (98% Milli-Q water, 2% methanol) and an organic mobile phase B (methanol). A gradient was applied to run from 100% A to 100% B over 30 minutes and remained at 100% B for 30 minutes and then the column was re-equilibrated in 100% A for 40 minutes. The eluent was then passed to the built-in fraction collector and the subsequent collected fractions were then 0.2-0.4 μm filtered to sterilise and assayed for antibacterial activity using the Turbidometric assay (TB) now described.

### Antimicrobial Assays

Bacteria were grown for 17 hours in 20 ml of tryptic soy broth (TSB). The bacteria were grown at 30° C. with the culture being oscillated at a rate of 90 oscillations per minute. Following growth, the bacteria were washed in phosphate buffered saline, pH 7.4, and adjusted to a concentration of 2×10⁵ bacteria per ml in fresh TSB.

To measure antibacterial activity, a turbidometric (TB) assay was carried out. One hundred and fifty microlitres of each HPLC fraction was mixed with 16.7 μl of 10% peptone water, pH 8.5, and 50 μl were then incubated with 10 μl of bacterial suspension in triplicate in the wells of a sterile, flat-bottom, 96-well microtitre plate. The samples were incubated at 37° C. for 24 hours and the optical density at 550 nm (OD₅₅₀) measured at hourly intervals from time zero. Controls replaced HPLC fractions with sterile ultrapure water.

All data points were measured against time zero. The survival index (SI) was then calculated as follows to express the growth of bacteria in the HPLC fractions as a percentage of growth in control samples.

$$SI = \frac{{OD}_{550} at mid \text{-} log of control bacterial growth}{{OD}_{550} of test samples at corresponding time point} \times 100$$

### Detection of Antimicrobial Activity

Antimicrobial activity was collected between 40.5-41.0 minutes, and further separation was conducted by injecting 100 μL onto a BioBasic SEC-60 HPLC column from Thermo Electron Corporation (U.K).

For this further separation of components, the mobile phase consisted of 100% Milli-Q water and the flow rate altered step-wise from 0.25 mL/min to 1.5 mL/min over 60 minutes. Fractions were collected using the built-in fraction collector and assayed for anti-bacterial activity.

Antibacterial activity against MRSA, S. aureus and E. coli (representative Gram +ve and Gram −ve bacteria) was seen in fractions collected between 44-48 minutes and these fractions were further examined by mass spectrometry.

Accurate mass determination was performed on a Q-ToF Ultima mass spectrometer (Micromass, Manchester, U.K) using a lockspray source. The fractions were introduced to the Q-ToF MS using a LC Packings Ultimate HPLC system (Dionex, Amsterdam, Netherlands). A FAMOS microautosampler equipped with a 1 μL loop and a full loop injection program was used for injections. From the FAMOS loop the samples were passed directly into the MS through fused silica tubing with the Chromeleon software suite used for instrument control and for the triggering of MS data acquisition.

Sample introduction was delivered with a mobile phase of 50% methanol in Milli-Q water and 0.1% formic acid. The reference standard used was 2 pmol/μL cytidine-5-monophosphate disodium salt.

The Q-ToF MS was operated in positive ionisation mode with a spray voltage of 3 kV, source temperature of 80° C., cone voltage of 35V, MCP voltage set to 2300V and ToF voltage of 9.10 kV. The collision energy was set to 4 for full scan spectra experiments and the automated data collection was controlled through the MassLynx program.

The isolated extract from the collection between 44-48 minutes was analysed and the empirical formula so obtained was C₁₀H₁₆N₆O₉.

Aspects of the invention are now described with reference to the following figures which are provided by way of example only. The figures show that the antimicrobial composition of the invention has the potential to act against a wide range of microbes.

FIG. 1 shows antimicrobial activity of <500 Da ultrafiltration fraction (containing seraticin) against a range of Gram+ve (stripe) and Gram−ve (black) bacteria and fungi (Candida albicans). Proteus mirabilis, MRSA 15, Staphylococcus epidermidis 8400 and MRSA 252 are all resistant to the antibacterial effects of the <500 Da fraction, however, this may be a concentration issue as further testing of MRSA 252 using purified seraticin (fraction 44-48, FIG. 4) demonstrated sensitivity to the compound. Bacterial growth is shown as a percentage of control growth.

FIG. 2 shows the antibacterial activity of seraticin-containing fractions (SC) from size exclusion chromatography (SEC) and C30 separations against S. aureus.

FIG. 3 shows the antibacterial activity of seraticin-containing fractions (SCF) from exclusion chromatography (SEC) and C30 separations against E. coli.

FIG. 4 shows the antibacterial activity of 44-48 min fraction against MRSA 252 and this shows clearly that the specific isolate of the empirical formula claimed has an improved antibacterial activity.

The invention covers not only individual embodiments as discussed but combinations of embodiments as well. It is to be understood that modifications and variations of the present invention will become apparent to those skilled in the art and it is intended that all such modifications will be included within the scope of the present invention. Further, the composition claimed when used as a medicament may be provided in the form of a salt, an ester or pro drug derivative.

1. An antimicrobial composition having the empirical formula of C₁₀H₁₆N₆O₉.

2. An antimicrobial composition according to claim 1 which is an isolate from invertebrates.

3. An antimicrobial composition according to claim 1 which is an isolate from the excretions/secretions of fly larvae.

4. An antimicrobial composition according to claim 3, wherein the fly larvae are of the species Lucilia sericata.

5. A treatment preparation including an antimicrobial composition according to claim 1.

6. A treatment preparation according to claim 5, including carrier composition for the antimicrobial composition.

7. A treatment preparation according to claim 6, wherein the carrier is selected from one or more of the following: water, saline, physiological saline, ointments, creams, oil in water emulsions, gels, solvents or combinations of solvents.

8. A treatment preparation according to claim 7, further including one or more of the following: a chelating agent, a pH buffering agent or a surfactant.

9. A treatment preparation according to claim 7 including one or more wound healing promoters.

10. A treatment preparation according to claim 9, wherein the wound healing promoter is Vitamin E.

11. A treatment preparation according to claim 5, including one or more anti-inflammatory- agents or agents that can reduce pain.

12. A wound dressing including an antimicrobial composition having the empirical formula of Ci₀Hi₆N₆O₉.

13. A wound dressing according to claim 12, wherein the antimicrobial composition is an isolate from the secretions/excretions of fly larvae.

14. A wound dressing according to claim 12, wherein the fly larvae is Lucilia.

15. A wound dressing according to claim 12, wherein the antimicrobial composition is included in a carrier composition for the antimicrobial composition.

16. A wound dressing according to claim 14, wherein carrier includes one or more of a pH buffering agent, a tissue growth promoting agent, an antiinflammatory or a pain killer and a surfactant.

17. An antiseptic composition including an antimicrobial composition of the formula Ci₀H₁₆N₆O₉.

18. A disinfectant composition including an antimicrobial composition of the formula Ci₀Hi₆N₆O₉.

19. A surface cleaner including an antimicrobial composition of the formula Ci₀Hi₆N₆O₉.

20. A method of treating an infection, said method comprising contacting an infected area with a therapeutic amount of an antimicrobial composition of the empirical formula Ci₀Hi₆N₆O₉ or via systemic administration of said antimicrobial composition.

21. A method according to claim 20, wherein the antimicrobial composition is included in a pharmaceutically acceptable carrier.

22. A method according to claim 21, wherein the carrier includes one or more of a pH buffering agent, a tissue growth promoting agent, an anti-inflammatory or a pain killer and a surfactant.

23. A method according to claim 20, wherein the infection is a wound infection.

24. A method according to claim 22, wherein the infection is due to MRSA.
